(19) United States
(12) Patent Application Publication
(10) Pub. No.: US 2006/0280714 A1
(43) Pub. Date: Dec. 14, 2006

(54) Aluminum starch octenylsuccinate derivative of small-granule wheat starch and cosmetic formulations made therefrom

(76) Inventors: Clodualdo C. Maningat, Platte City, MO (US); Sukh D. Bassi, Atchison, KS (US); Kyungsoo Woo, Shawnee, KS (US); Dharmen B. Makwana, Platte City, MO (US)

Correspondence Address: LATHROP & GAGE LC, 4845 PEARL EAST CIRCLE, SUITE 300, BOULDER, CO, 80301, US

(21) Appl. No.: 11/387,419

(22) Filed: Mar. 23, 2006

Related U.S. Application Data
(60) Provisional application No. 60/664,433, filed on Mar. 23, 2005.
(60) Provisional application No. 60/664,814, filed on Mar. 24, 2005.

(51) Int. Cl. A61K 8/73; C08B 33/02

(52) U.S. Cl. 424/70.13; 536/110

(57) ABSTRACT

A small-granule aluminum starch octenylsuccinate (ASO) derivative having a distribution of starch granules with a mean diameter of between 4.5 μm and 8.9 μm is disclosed herein. The small-granule ASO may be used in a wide variety of personal care formulations.

### RELATED APPLICATIONS

This application claims the benefit of priority to U.S. provisional patent application Ser. No. 60/664,433, filed Mar. 23, 2005 and U.S. provisional patent application Ser. No. 60/664,814, filed Mar. 24, 2005, each of which is incorporated herein by reference.

### FIELD OF THE INVENTION

The present invention generally pertains to an aluminum starch octenylsuccinate derivative of small-granule wheat starch, and cosmetic formulations including the aluminum starch octenylsuccinate derivative of small-granule wheat starch.

### BACKGROUND

Starch is the predominant carbohydrate component of most cereals, legumes, tubers and root crops. It is deposited in plants as tiny, partially crystalline granules. The sizes, shapes, amylose contents and gelatinization temperatures of starch granules are affected by the botanical source of starch. In wheat, two distinct classes of granules differing in size and shape have been reported by Seib, P.S. Wheat Starch: Isolation, Structure, and Properties. Oyo Toshitsu Kagaku 41: 49-69 (1994) and Cornell, H.J. and Hoveling, A.W. Wheat Chemistry and Utilization. Technomic Publishing, Lancaster, Pa. (1998). The two classes are small (˜5 μm), spherical granules and large (˜20 μm), disk-shaped granules.

Personal care products usually contain starch in the form of aluminum starch octenylsuccinate (ASO), which is the aluminum salt of the reaction product of 1-octenylsuccinic anhydride with starch; see, e.g., U.S. Pat. Nos. 2,613,206 and 2,661,349. ASO is not an ocular irritant and does not produce abnormal skin reactions in guinea pigs and rabbits (Anonymous “Final report on the safety assessment of aluminum starch octenyl succinate”, Int. J. Toxicol. 21: 1-7. (2003)). It is nontoxic to rats by inhalation, and clinical facial sting, chamber scarification, and closed patched studies indicated little irritation potential when ASO was tested up to 3% in cosmetic formulations. ASO is used in cosmetic products up to 30% as an anti-caking agent and as a nonaqueous thickening agent. Guth et al. “Polymeric approaches to skin protection”, Cosmet. Toiletries 106: 71-74 (1991) described the performance of aluminum starch octenylsuccinate as an aesthetics control agent in creams and lotions, where it may be used to mitigate the greasiness and drag often associated with products containing high levels of occlusive agents or organic-based UV absorbers. ASO has been reported to enhance the sun protection factor of sunscreen formulations. For example, the sun protection factor value of titanium dioxide in a formulation can be enhanced by as much as 40% upon addition of as little as 5% aluminum starch octenylsuccinate.

Commercially-available wheat starches contain both small (˜5 μm), spherical granules and large (˜20 μm), disk-shaped granules. When wheat starch is used in personal care products, the non-uniform size and morphology within the granular mixture can impact the properties and performance of the final product. For example, products made from wheat starch having a normal distribution of size and shape may not spread easily, may not provide a smooth finish and/or may not effectively absorb oil.

### SUMMARY

The present instrumentalities advance the art and overcome the problems that are outlined above.

In one aspect, aluminum starch octenylsuccinate (ASO) has a distribution of starch granules with a mean diameter of between 4.5 μm and 8.9 μm. The personal care products including the small-granule ASO may be selected from the group consisting of conditioning shampoo, damaged hair shampoo, hand cream, sunscreen lotion, liquid talc, a soap bar, lipstick, liquid make-up, and a mud mask.

In one aspect a small-granule starch composition includes a plurality of starch granules having a size distribution with a mean diameter between 4.5 μm and 8.9 μm, the starch granules modified by reaction with 1-octenylsuccinic anhydride and an aluminum salt.

### BRIEF DESCRIPTION OF THE DRAWINGS

FIG. 1 shows a scanning electron micrograph (SEM) (1000x) of unmodified small-granule wheat starch.

FIG. 2 shows a scanning electron micrograph (SEM) (1000x) of unmodified normal-granule wheat starch.

FIG. 3 shows a scanning electron micrograph (SEM) (1000x) of small-granule aluminum starch octenylsuccinate.

FIG. 4 shows a scanning electron micrograph (SEM) (1000x) of normal-granule aluminum starch octenylsuccinate.

### DETAILED DESCRIPTION

The present instrumentalities overcome the above described deficiencies and provide an aluminum starch octenylsuccinate derivative from wheat with a small average particle size. The small-granule ASO derivative has a smooth feel, good slip, and high oil absorption capacity. In one embodiment, cosmetic products may be formulated to contain from 0.1-15% small-granule ASO wheat starch.

The following examples set forth the preparation of the aluminum starch octenylsuccinate derivative of small-granule wheat starch and the formulation of this starch derivative in several cosmetic products. It is to be understood, however, that these examples are provided by way of illustration, and not by limitation, to illustrate preferred embodiments of what is claimed.

### EXAMPLE 1

### Preparation and Characterization of Aluminum Starch Octenylsuccinate Derivative of Small-Granule Wheat Starch

Starch Modification. Commercially available small-granule wheat starch (brand name Puramyl SP) was purchased from Latenstein BV (Netherlands). The starch was dispersed in water and modified with 1-octenylsuccinic anhydride and aluminum sulfate according to the provisions of the U.S. Code of Federal Regulations (Title 21 CFR §172.892) for Food Starch-Modified.

Starch Characterization. Granule size distribution was determined using a Coulter MultiSizer II (Beckman Coulter, Inc., Miami, Fla.). Using number statistics, the small-granule wheat starch (Puramyl SP) shows a mean diameter of 4.5 μm. By volume statistics, the mean diameter is 8.9 μm. In contrast, normal wheat starch (brand name Midsol 50 from MGP Ingredients, Inc., Atchison, Kans.) has a mean diameter of 10.6 and 18.1 μm by number statistics and volume statistics, respectively.

TABLE 1
Mean diameter of small-granule wheat starch and normal-granule
wheat starch.

| | Mean Diameter by | Mean Diameter by |
|---|---|---|
| Starch Samples | Number Statistics | Volume Statistics |
| Small-Granule | 4.5 μm | 8.9 μm |
| Wheat Starch | | |
| Normal-Granule | 10.6 μm | 18.1 μm |
| Wheat Starch | | |

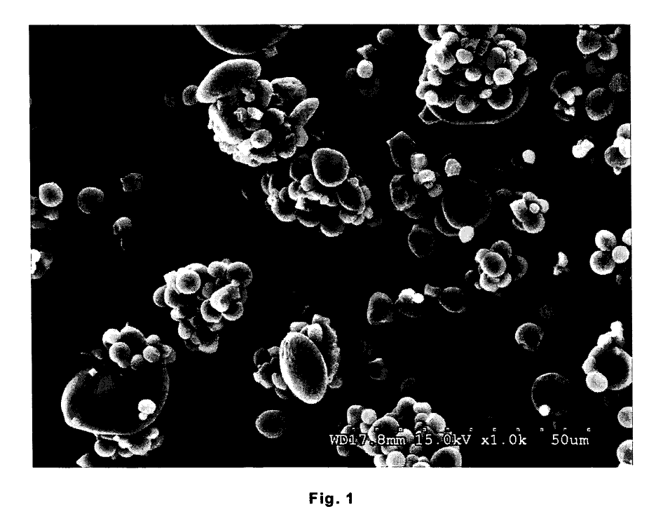

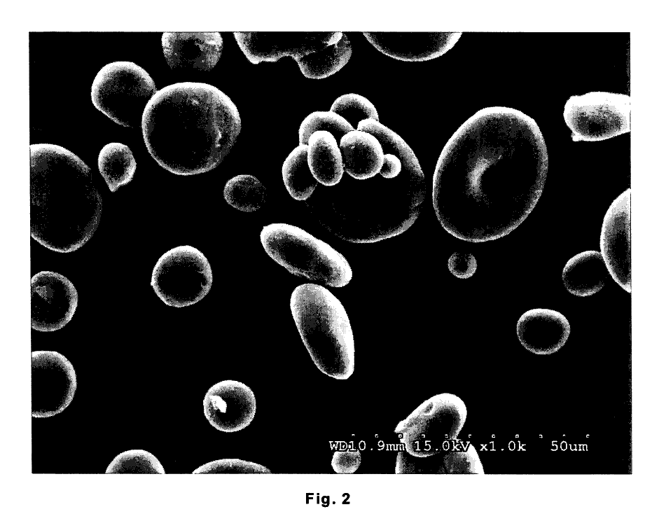

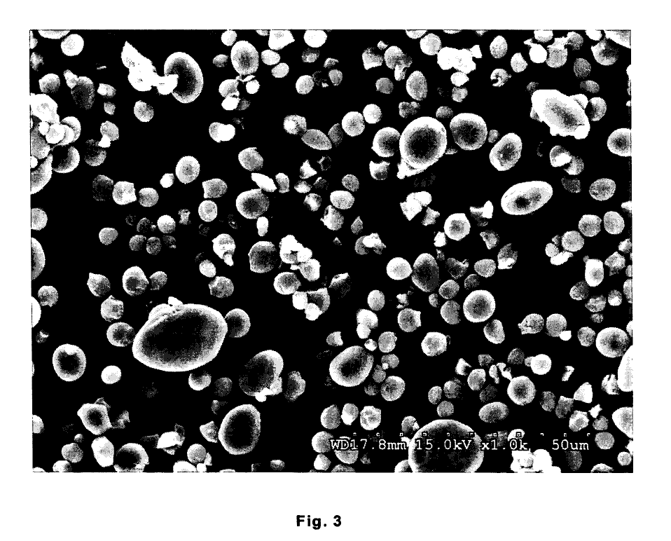

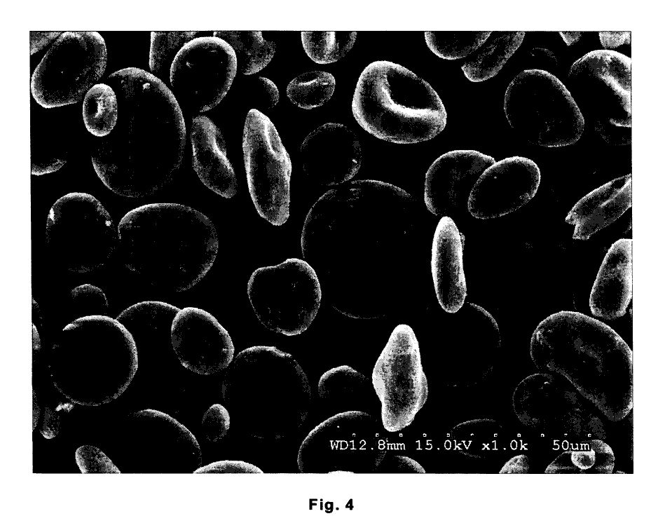

Surface topography of starch granules was determined using a scanning electron microscope. Samples were gold coated with a Hummer Sputter Coating System and imaged with a Hitachi 3000N Scanning Electron Microscope. A comparison of the appearance of small-granule and normal-granule wheat starch is shown in FIGS. 1 and 2, respectively. A predominance of small, spherical granules is evident in FIG. 1, whereas both the large, disk-shaped and small, spherical granules appear in FIG. 2. After modification of small-granule wheat starch, the aluminum starch octenylsuccinate derivative (brand name Skin Flow Ultra) exhibits the typical size and shape shown in FIG. 3. Similarly, modification of normal-granule wheat starch to yield the aluminum starch octenylsuccinate derivative (brand name Skin Flow) shows the typical size and shape shown in FIG. 4. Oil absorption capacity was determined by following the procedure in ASTM D 281-95. The oil absorption capacity of the aluminum starch octenylsuccinate derivative of small-granule wheat starch is 0.45 gram oil per gram of the small-granule wheat ASO versus 0.41 gram oil per gram of the normal-granule wheat ASO. Water repellency was evaluated by adding 5 grams of starch sample to 75 ml of water in a graduated flask, gently mixing by inverting the flask ten times, and allowing the mixture to sit undisturbed for 1 hour. The mixture was mixed a second time by inverting the flask ten times and then allowed to sit undisturbed for 15 minutes. Water repellency after 15 minutes was determined by measuring the volume of sedimented starch in the flask. The water repellency of the ASO derivative of small-granule wheat starch was <0.1 ml, whereas the water repellency of normal-granule wheat ASO was 7 ml. When applied to the skin, the ASO derivative of small-granule wheat starch appears to have a smoother, more velvety feel than the ASO derivative of normal-granule wheat starch.

### EXAMPLE 2

PREPARATION OF CONDITIONING SHAMPOO

| | Trade Name | INCI Name | W/W % |
|---|---|---|---|
| A | Water | Aqua | Adjust |
| A | ETD 2020 | Acrylate/C10-30 Alkyl | 0.20 |
| | | Acrylate Crosspolymer | |
| A | PK-771 | PVP | 2.80 |
| A | Merquat 550 | Polyquaternium-7 | 0.10 |
| A | Versene NA2 | Disodium EDTA | 0.10 |
| A | Jeechem GC-7 | PEG-7 Glycerox HE | 0.20 |
| A | Stepanol CS-230 | Sodium Laureth Sulfate | 25.00 |
| A | Stepanol WAC | Sodium Lauryl Sulfate | 10.00 |
| A | Mackham 35 | Cocomidopropyl Betaine | 2.50 |
| A | Jojoba Oil Yellow | Simmondsia Chinensis | 0.50 |
| A | Almond Oil | Prunus Amygdalus Dulcis | 0.10 |
| A | Skin Flow Ultra | Aluminum Starch | 0.50 |
| | | Octenylsuccinate | |
| A | Glycerin USP | Glycerol | 2.00 |
| B | D-L Panthenol | Panthenol | 0.05 |
| B | Aloe Vera Extract | Aloe Barbadensis | 0.03 |
| B | Rosehip Extract | Rosa Canina | 0.06 |
| C | Preservative | Propylene Glycol and | 0.40 |
| | | Diazolidinyl Urea and | |
| | | Methylparaben and | |
| | | Propylparaben | |
| C | Fragrance | Fragrance | 0.10 |
| C | Blue # 1 | FD&C Blue # 01 | QS |

QS = quality standard

In a suitable primary tank, the required amount of distilled water was metered out. Part A ingredients were added to the primary tank in the order listed. The Part B ingredients were then added to the primary tank. The Part C ingredients were added to the primary tank in the order listed. The pH was adjusted to 5.5-6.5 (25% AMP 95) and the viscosity was adjusted with NaCI to the desired thickness.

### EXAMPLE 3

PREPARATION OF DAMAGED HAIR SHAMPOO

| | Trade Name | INCI Name | W/W % |
|---|---|---|---|
| A | Water | Aqua | Adjust |
| A | ETD 2020 | Acrylate/C10-30 Alkyl | 0.20 |
| | | Acrylate Crosspolymer | |
| A | PK-771 | PVP | 2.80 |
| A | Jeechem GC-7 | PEG-7 Glycerox HE | 0.20 |
| A | Glycerin | Glycerol | 1.00 |
| A | Jeelate ES-2 | Sodium Laureth Sulfate | 25.00 |
| A | Stepanol WAC | Sodium Lauryl Sulfate | 10.00 |
| A | Mackham 35 | Cocomidopropyl Betaine | 2.50 |
| A | Jojoba Oil Yellow | Simmondsia Chinensis | 0.50 |
| A | Almond Oil | Prunus Amygdalus Dulcis | 0.10 |
| A | Skin Flow Ultra | Aluminum Starch | 0.50 |
| | | Octenylsuccinate | |
| B | D-L Panthenol | Panthenol | 0.05 |
| C | Preservative | Propylene Glycol and | 0.40 |
| | | Diazolidinyl Urea and | |
| | | Methylparaben and | |
| | | Propylparaben | |
| C | Aqua Pro ® II WP | Hydrolyzed Wheat Protein | 1.50 |
| C | Fragrance | Fragrance | 0.10 |
| C | Blue # 1 | FD&C Blue # 01 | QS |

QS = quality standard

In a suitable primary tank, the required amount of distilled water was metered out. Part A ingredients were added to the primary tank in the order listed. The Part B ingredients were added to the primary tank. The Part C ingredients were added to the primary tank in the order listed. The pH was adjusted to 5.5-6.5 (25% AMP 95) and the viscosity was adjusted with NaCI to the desired thickness.

### EXAMPLE 4

PREPARATION OF HAND CREAM

| | Trade Name | INCI Name | W/W % |
|---|---|---|---|
| A | Water | Aqua | QS |
| A | Glycerin | Glycerin | 1.50 |
| A | Skin Flow Ultra | Aluminum Starch | 2.00 |
| | | Octenylsuccinate | |
| B | Jeen Stearic Acid | Stearic Acid | 3.00 |
| B | Lipo GMS 450 | Glyceryl Sterate | 2.00 |
| B | Lipocol C | Cetyl Alcohol | 2.00 |
| B | Promulgin D | Cetearyl Alcohol and | 1.00 |
| | | Ceteareth-20 | |
| B | Lipocol S | Steraryl Alcohol | 1.00 |
| B | Coconut Oil | Theobromo Cacao | 0.50 |
| B | Lipo IPM | Isopropyl Myristate | 0.50 |
| B | Lipo IPP | Isopropyl Palmitate | 0.50 |
| B | Jojoba Oil | Simmondsia Chinensis | 0.75 |
| B | Nipagin M | Methylparaben | 0.20 |
| B | Nipasol M | Propylparaben | 0.15 |
| C | Dow Corning 200 | Dimethicone | 0.50 |
| | Fluid | | |
| C | Phenoxyethanol | Phenoxyethanol | 0.30 |
| D | Fragrance | Fragrance | QS |

Part A ingredients were added to a primary tank and heated to 75° C. In a secondary tank, the Part B ingredients were heated to 75° C. Part B was added to Part A. With good mixing, the Part C ingredients were added to the batch at 65° C. The Part D ingredients were added to the batch when the temperature reached 35° C.

### EXAMPLE 5

PREPARATION OF SUNSCREEN MOISTURIZING LOTION

| | Trade Name | INCI Name | W/W % |
|---|---|---|---|
| A | Water | Aqua | Adjust |
| A | Versene NA | Disodium EDTA | 0.10 |
| A | Acritamer 940 | Carbomer | 0.15 |
| A | AMP 95 Regular | 2-Amino-2-Methyl-1-Propanol | QS |
| B | Jeen Stearic Acid | Stearic Acid | 2.00 |
| B | Promulgin D | Cetearyl Alcohol and | 1.50 |
| | | Ceteareth-20 | |
| B | Liponate SPS | Cetyl Ester | 1.50 |
| B | Lipovol MS-70 | Mineral Oil | 0.50 |
| B | Lipo GMS 450 | Glyceryl Sterate | 2.50 |
| B | Dow Corning 200-350 ct | Dimethicone | 0.50 |
| B | Jeescreen OMC | Ethylhexyl Methoxycinnamate | 7.00 |
| C | Finsol TN | C12-15 Alkyl Benzoate | 2.75 |
| C | Skin Flow Ultra | Aluminum Starch | 2.00 |
| | | Octenylsuccinate | |
| D | Liquapar PE | Phenoxyethanol (and) | QS |
| | | Isopropylparaben (and) | |
| | | Isobutylparaben (and) | |
| | | Butylparaben | |
| D | Fragrance | Fragrance | 0.05 |

In a suitable primary tank, the required amount of distilled water was metered out. Part A ingredients were added to the primary tank and heated to 75° C. In a secondary tank, the Part B ingredients were weighed out in the order listed and heated to 75° C. Part B was added to Part A. Part C was premixed and added to the batch at 70° C. The Part D ingredients were added to the batch when the temperature reached 40° C.

### EXAMPLE 6

PREPARATION OF REFRESHING LIQUID TALC (LOTION)

| | Trade Name | INCI Name | W/W % |
|---|---|---|---|
| A | Water | Aqua | 67.4 |
| A | Glycerin USP | Glycerin | 1.00 |
| B | Lipocol C | Cetyl Alcohol | 1.50 |
| B | Jeen Stearic Acid | Stearic Acid | 1.50 |
| B | Lopocol S | Stearyl Alcohol | 0.50 |
| B | Promulgin D | Cetearyl Alcohol and | 0.80 |
| | | Ceteareth-20 | |
| B | Rita GMS | Glyceryl Sterate | 0.50 |
| B | Kessco IPP | Isopropylpalmitate | 1.00 |
| B | Dow Corning-200 | Dimethicone | 0.20 |
| C | Skin Flow Ultra | Aluminum Starch | 3.00 |
| | | Octenylsuccinate | |
| C | Lipo IPM | Isopropyl Myristate | 2.00 |
| D | SD-40 | Alcohol | 20.0 |
| D | Fragrance | Fragrance | 0.20 |
| | (Plumeria) | | |
| D | Liquapar PE | Phenoxyethanol (and) | 0.40 |
| | | Isopropylparaben (and) | |
| | | Isobutylparaben (and) | |
| | | Butylparaben | |

QS = Quality Standard

The Part A ingredients were added to a primary tank and heated to 75° C. In a secondary tank, the Part B ingredients were mixed and heated to 75° C. Part B was added to Part A. Part C was premixed and added to the batch at 55° C. The Part D ingredients were added to the batch in the order listed when the temperature reached 35° C.

### EXAMPLE 7

PREPARATION OF MOISTURIZING SOAP BAR

| | Trade Name | INCI Name | Grams |
|---|---|---|---|
| A | Almond Oil | Pruns Amygdalus Dulcis | 2.00 |
| A | Canola Oil | Canola Oil | 10.00 |
| A | Coconut Oil | Theobromo Cacao | 20.00 |
| A | Jojoba Oil | Simmondsia Chinensis | 3.00 |
| A | Mango Oil | Mangifera Indica | 3.00 |
| A | Palm Kernel Oil | Elaeis Guineensis Oil | 10.00 |
| A | Safflower Oil | Carthamus Tinctorius | 12.00 |
| A | Soybean Oil | Glycine Soja Oil | 7.00 |
| A | Sunflower Oil | Helianthus Annuus Oil | 4.00 |
| B | Sodium Hydroxide¹ | Sodium Hydroxide | 20.00 |
| B | Water | Aqua | 19.00 |
| B | Skin Flow Ultra | Aluminum Starch | 5.00 |
| | | Octenylsuccinate | |
| C | Foam Pro L | Hydrolyzed Wheat Protein | 4.00 |
| D | Fragrance | Fragrance | 3.00 |
| D | Color | Color | QS |

QS = quality standard
¹Sodium Hydroxide 50%

In a suitable primary tank, the Part A ingredients were mixed and heated to 45° C. In a secondary tank, the Part B ingredients were premixed and combined with Part A. The mixture was stirred until tracing was observed (˜20-25 minutes). Part C was added to the batch and stirred. Part D was added to the batch and stirred. The soap was poured into molds and then covered with a warm blanket for 24 hours. The soap was removed from the molds and allowed to cure for a few days.

### EXAMPLE 8

PREPARATION OF MOISTURIZING LIPSTICK

| | Trade Name | INCI Name | W/W % |
|---|---|---|---|
| A | Castor Oil | Ricinus Communis | Adjust |
| A | Candelilla Wax | Euphorbia Cerfera | 4.50 |
| A | Carnauba Wax | Copernicia Cerfera | 1.50 |
| A | Ozokerite Wax | Ozokerite | 1.25 |
| A | Microcrystalline | Microcrystalline Wax | 2.50 |
| | Wax | | |
| B | Liponate CG | Caprylic Capric Triglyceride | 12.00 |
| B | Lipovol MOS-70 | Mineral Oil | 1.50 |
| B | Jeechem OOS | Octyldodecyl Stearoyl Sterate | 4.00 |
| B | Lipo IPM | Isopropyl Myristate | 1.00 |
| B | Lipo IPP | Isopropyl Palmitate | 1.00 |
| B | Tocopheryl Acetate | Tocopheryl Acetate | 0.05 |
| C | Yellow Iron Oxide | Iron Oxide Yellow | QS |
| C | Black Iron Oxide | Iron Oxide Black | QS |
| C | Red Iron Oxide | Iron Oxide Red | QS |
| D | Castor Oil | Ricinus Communis | 5.00 |
| D | Skin Flow Ultra | Aluminum Starch | 2.00 |
| | | Octenylsuccinate | |
| E | Nipagin M | Methylparaben | 0.20 |
| E | Nipasol M | Propylparaben | 0.15 |
| E | Fragrance | Fragrance | QS |

Part A ingredients were added to a tank and heated to 75° C. In a secondary tank, the Part B ingredients were heated to 75° C. Part B was added to Part A. Part C and Part D were premixed and then added to the A-B mixture at 75° C. The temperature was maintained at 75° C until the color was uniform. The preservative and fragrance were added and then the mixture was set in molds at 45° C.

### EXAMPLE 9

PREPARATION OF LIQUID MAKE-UP

| | Trade Name | INCI Name | W/W % |
|---|---|---|---|
| A | Deionized Water | Aqua | Adjust |
| A | Triethanolamine | Triethanolamine | QS |
| | (99%) | | |
| A | Propylene Glycol | | 5.00 |
| | (Universal) | | |
| A | Carbomer 940 | Carbomer | 0.20 |
| A | Veegum | | 0.30 |
| A | Skin Flow Ultra | Aluminum Starch | 2.00 |
| | | Octenylsuccinate | |
| B | Stearic Acid (Lipo) | | 1.00 |
| B | PEG-120 Dimethyl | | 1.00 |
| | Glucose | | |
| | Sesquesterate | | |
| B | Stearate-20 | | 0.80 |
| B | Cetyl Alcohol | | 0.50 |
| B | Ceteth-20 | | 0.50 |
| B | Methyl Glucose | | 1.00 |
| | Sesquesterate | | |
| B | Nylon-12 (Lipo) | | 3.50 |
| C | Isopar-G | | 0.20 |
| C | Isopar-M | | 0.20 |
| D | Silica Dimethyl | | 1.00 |
| | Siliylate (Cabot) | | |
| D | Cyclomethicone | | 15.00 |
| | (Dow Corning) | | |
| E | Titanium Dioxide | | 2.00 |
| | (Rona) | | |
| E | Iron Oxide Yellow | | 0.40 |
| | (BF Goodrich) | | |
| E | Iron Oxide Black | | 0.20 |
| | (BF Goodrich) | | |
| E | Iron Oxide Red (BF | | 0.30 |
| | Goodrich) | | |
| F | Polysorbate-20 | | 1.00 |
| G | Preservative | | QS |
| H | Fragrance | | QS |

Part A ingredients were added to a tank and the mixture was heated to 75° C. Part E ingredients were pressed through a colloid mill with some propylene glycol and recirculated until the pigments were evenly dispersed. The colloid mill was rinsed with Part C and a lightning mixer was used to thoroughly mix the ingredients, which were heated to 75° C. In a secondary tank, the Part B ingredients were premixed and heated to 75° C. Part B was added to the main batch. Part E was added to the main batch. Part D was premixed and then added to the main batch when the temperature reached 65° C. Part F was added to the main batch when the temperature reached 40° C. The preservative and fragrance were added. The pH and viscosity were adjusted to desired levels.

### EXAMPLE 10

PREPARATION OF REFRESHING MUD MASK

| | Trade Name | INCI Name | W/W % |
|---|---|---|---|
| A | Water | Aqua | Adjust |
| A | Carbomer 940 | Carbomer | 0.15 |
| A | Triethanolamine | Triethanolamine | QS |
| | (99%) | | |
| A | Skin Flow Ultra | Aluminum Starch | 1.00 |
| | | Octenylsuccinate | |
| B | Propylene Glycol | Propylene Glycol | 5.00 |
| B | L-Menthol | L-Menthol | 0.10 |
| C | China Clay | Clay | 12.00 |
| C | Eusolex T-200 | Titanium Dioxide | 0.50 |
| D | Preservative | Preservative | QS |
| D | Aqua Pro ® WP | Hydrolyzed Wheat Protein | 1.50 |
| D | Aqua Pro ® WAA | Wheat Amino Acid | 0.50 |
| D | Fennel Extract | Fennel Extract | 0.50 |

Part A ingredients were added to a tank and mixed until a uniform solution resulted. The solution was then heated to 75° C. and cooling was started. When the temperature reached 45° C., the Part C ingredients were sprinkled into Part A in the order listed. Part B was added to the batch. The ingredients of Part D were premixed and added to the batch.

Changes may be made in the above compositions and methods without departing from the subject matter described in the Summary and defined by the following claims. It should thus be noted that the matter contained in the above description should be interpreted as illustrative and not limiting.

All references cited are incorporated by reference herein.

1. Aluminum starch octenylsuccinate (ASO) having a number mean diameter of about 4.5 μm.

2. The ASO of claim 1 further comprising a water repellency less than about 0.1 ml of sedimented starch per 5 grams of sample dispersed in 75 ml water.

3. The ASO of claim 1 further comprising a oil absorption capacity in excess of about 0.45 grams of oil per gram of sample.

4. A personal care product including the ASO of claim 1.

5. A personal care product comprising from 0.1 -10% by weight of the ASO of claim 1.

6. The personal care product of claim 4, wherein the personal care product is selected from the group consisting of conditioning shampoo, damaged hair shampoo, hand cream, sunscreen lotion, liquid talc, a soap bar, lipstick, liquid make-up, and a mud mask.

7. A personal care product comprising from 0.5-10% by weight of the ASO of claim 1.

8. A small-granule starch composition comprising:
a plurality of starch granules having a number mean diameter of about 4.5 μm, said starch granules modified by reaction with 1-octenylsuccinic anhydride and an aluminum salt.

9. A personal care product including the composition of claim 8.

10. A personal care product comprising from 0.1-10% by weight of the composition of claim 8.

11. The personal care product of claim 9, wherein the personal care product is selected from the group consisting of conditioning shampoo, damaged hair shampoo, hand cream, sunscreen lotion, liquid talc, a soap bar, lipstick, liquid make-up, and a mud mask.

12. A personal care product comprising from 0.5-10% by weight of the composition of claim 8.

13. The composition of claim 8, wherein said starch granules further comprise a volume mean diameter of about 8.9 μm.

14. A composition comprising aluminum starch octenylsuccinate (ASO) having a volume mean diameter of about 8.9 μm.

15. The ASO of claim 14 further comprising a water repellency less than about 0.1 ml of sedimented starch per 5 grams of sample dispersed in 75 ml water.

16. The ASO of claim 14 further comprising a oil absorption capacity in excess of about 0.45 grams of oil per gram of sample.

17. A personal care product including the ASO of claim 14.

18. The personal care product of claim 17, wherein the personal care product is selected from the group consisting of conditioning shampoo, damaged hair shampoo, hand cream, sunscreen lotion, liquid talc, a soap bar, lipstick, liquid make-up, and a mud mask.

19. A personal care product comprising from 0.1-10% by weight of the ASO of claim 14.

20. A personal care product comprising from 0.5-10% by weight of the ASO of claim 14.
